(19) United States
(12) Patent Application Publication
(10) Pub. No.: US 2025/0041229 A1
(43) Pub. Date: Feb. 6, 2025

(54) SUPPLEMENTAL COATING AND RELATED METHOD

(71) Applicant: Nutramax Laboratories, Inc., Lancaster, SC (US)

(72) Inventors: David GRIFFIN, Lancaster, SC (US); Binh DAVIS, Lancaster, SC (US); Hubert RATKOWSKI, Lancaster, SC (US)

(21) Appl. No.: 18/717,197

(22) PCT Filed: Dec. 7, 2022
(86) PCT No.: PCT/US2022/081071

§ 371 Date: Jun. 6, 2024

Related U.S. Application Data
(60) Provisional application No. 63/287,114, filed on Dec. 8, 2021.

(51) Int. Cl. A61K 9/28; A61K 31/7076; A61K 36/28

(52) U.S. Cl. CPC A61K 9/282; A61K 9/2813; A61K 9/2866; A61K 9/2893; A61K 31/7076; A61K 36/28

(57) ABSTRACT

A nutritional supplemental for a human or animal, such as a dog or a cat, which includes a partially hydrophobic coating applied to a tablet, is provided. The tablet includes SAMe (S-Adenosylmethionine) milk thistle, and/or any other hydroscopic raw material. SAMe is typically used as an active ingredient to support the liver of the animal. The coating of the tablet typically includes a polymer or resin, stearic acid as a moisture barrier, and calcium carbonate. The coating may also include a plasticizer and color agent. The polymer or resin is typically hydroxypropyl methyl cellulose, and the plasticizer typically includes triethyl citrate and optionally lecithin.

### CROSS REFERENCE TO RELATED APPLICATIONS

This application claims priority to and the benefit of international (PCT) patent application no. PCT/US2022/081071, filed Dec. 7, 2022, which claims the benefit of U.S. provisional patent application No. 63/287,114, filed Dec. 8, 2021, the entire contents of which is incorporated herein by reference.

### TECHNICAL FIELD

The disclosure relates generally to a supplement including a coating, such as a human or animal supplement, and methods of manufacturing the same.

### BACKGROUND

A nutritional supplement for oral administration to a human or animal, such as a dog, cat, or horse, can be provided in the form of a tablet. SAMe (S-Adenosylmethionine) is an example of a nutritional supplement that can be provided in the tablet. SAMe is known for supporting the liver of an animal and keeping the liver working properly. More specifically, SAMe has been shown to markedly increase liver levels of glutathione, an important compound for liver health. The glutathione is formed when SAMe is metabolized. SAMe can also help protect liver cells from cell death and may help cell repair and regeneration. SAMe has also been administered to animals to treat osteoarthritis, behavior changes, and other cognitive and nerve disorders.

The tablet containing SAMe typically includes an enteric coating to delay absorption of the SAMe in the animal. The enteric coating also provides protection from the environment. The enteric coating is stable at an acidic pH found in the stomach, but dissolves above a pH of 5 to 6. Thus, the enteric coating delays release of the SAMe until the tablet enters an area of higher pH, for example in the small intestine of the animal.

Enteric coatings are typically formed of polymers or other materials that have properties similar to polymers. Materials that have been used to form enteric coatings include resins, fatty acids, waxes, shellac, plastics, plant fibers, and organic solvents. One example material used to form the enteric coating is methacrylic acid. However, tablets containing SAMe and coated with the methacrylic acid tend to slowly absorb moisture when exposed to normal room temperature and when exposed to humidity (25° C. at 60% RH) at a rate that is faster than desired. Moisture penetrating the tablet causes the core of the tablet to expand, and then results in discharge or oozing of the contents of the tablet, or softening of the core of the tablet. Coated tablets containing SAMe which remain firm to the touch when exposed to such humidity is desired.

### SUMMARY

One aspect of the disclosure provides a coated tablet for humans or animals. The tablet may include SAMe, milk thistle, and/or any other hydroscopic raw material.

According to one embodiment, the coating of the coated tablet includes a polymer or resin, stearic acid, and calcium carbonate.

According to another embodiment, the coating of the coated tablet includes a polymer or resin, a moisture barrier, calcium carbonate, and a plasticizer.

Another aspect of the disclosure provides a method of manufacturing a coated tablet containing SAMe, milk thistle, and/or any other hydroscopic raw material. The method includes applying a coating to an uncoated tablet containing the SAMe, milk thistle, and/or any other hydroscopic raw material.

According to one embodiment, the coating applied to the uncoated tablet includes a polymer or resin, stearic acid, and calcium carbonate.

According to another embodiment, the coating applied to the uncoated includes a polymer or resin, a moisture barrier, calcium carbonate, and a plasticizer.

### DETAILED DESCRIPTION

One aspect of the disclosure provides a coated tablet containing a hydroscopic raw material, for example SAMe (S-Adenosylmethionine), which is a nutritional supplement for oral administration to a human or animal, such as a dog, cat, or horse. The SAMe is typically administered to animals to support the liver and keep the liver working properly. More specifically, SAMe has been shown to markedly increase liver levels of glutathione, an important compound for liver health. The glutathione is formed when SAMe is metabolized. SAMe can also help protect liver cells from cell death and may help cell repair and regeneration. SAMe may also be administered to animals to treat osteoarthritis, behavior changes, and other cognitive and nerve disorders.

According to example embodiments, the tablets include approximately 90 mg to approximately 675 mg of the SAMe (S-Adenosylmethionine) ion as an active ingredient. For example, the tablet can include approximately 90 mg of the SAMe ion, 225 mg of the SAMe ion, 425 mg of the SAMe ion, or 675 mg of the SAMe ion.

Various forms of the SAMe can be present in the tablet. For example the tablet can include a SAMe salt, such as S-Adenosyl Methionine (SAMe) Disulfate Tosylate, 1,4-butanedisulfonate, tosylate, or disulfate ditosylate.

In addition to the SAMe, the tablet may include other active ingredients and/or inactive ingredients. A variety of different ingredients can be used depending on the intended purpose of the tablet. According to one example embodiment, in addition to the SAMe, the tablet may further includes silybin. Silybin is an active part of an extract from milk thistle, which has been shown to support liver function by helping to maintain a healthy oxidative balance.

According to an example embodiment, the tablet includes approximately 90 mg of the SAMe ion and approximately 31 mg of silybin-phosphatidylcholine complex (SPC), providing 9 mg of silybin A+B. According to another embodiment, the tablet includes approximately 225 mg of the SAMe ion and approximately 82 mg of Silybin-phosphatidylcholine complex (SPC), providing 24 mg of silybin A+B. According to another embodiment, the tablet includes approximately 425 mg of the SAMe ion and approximately 120 mg of silybin-phosphatidylcholine complex (SPC), providing 35 mg of silybin A+B. According to another embodiment, the tablet includes approximately 675 mg of the SAMe ion and approximately 190 mg of silybin-phosphatidylcholine complex (SPC), providing 56 mg of silybin A+B.

According to an example embodiment, the coating includes at least a polymer or resin (also referred to as a polymer film former), a moisture barrier, and calcium carbonate. The moisture barrier is preferably stearic acid, such as purified stearic acid. The stearic acid can be present in an amount of 0.6 to 2.4 wt. %, based on the total weight of the uncoated tablet. The calcium carbonate can be present in an amount of 0.47 to 1.92 wt. %, based on the total weight of the uncoated tablet. It is believed that the combination of the stearic acid and the calcium carbonate provide a significant contribution to the beneficial properties of the coating, including stability and moisture resistance.

The coating may include various other ingredients. According to another embodiment, the coating includes at least a polymer or resin (also referred to as a polymer film former), a moisture barrier, calcium carbonate, and a plasticizer. The coating may also include an opacifying agent and/or a color agent. According to an example embodiment, the polymer film former is hydroxypropyl methyl cellulose, the moisture barrier is purified stearic acid, and the plasticizer is triethyl citrate and lecithin, for example sunflower lecithin. The lecithin is optional according to the example embodiment. The calcium carbonate may also serve as an opacifying agent. The coating according to this example embodiment also includes caramel. However, the coating may include various other ingredients.

According to example embodiments of the coating, the polymer or resin, for example hydroxypropyl methyl cellulose, is present in an amount of 0.7 to 2.8 weight percent (wt. %); the moisture barrier, for example purified stearic acid, is present in an amount of 0.6 to 2.4 wt. %; the calcium carbonate is present in an amount of 0.47 to 1.92 wt. %; the plasticizer, for example triethyl citrate and optionally lecithin, is present in a total amount of 0.2 to 0.8 wt. %; and the color agent, for example caramel color, is present in an amount of 0.02 to 0.09 wt. %, based on the total weight of the uncoated tablet (also referred to as the weight gain of the coating). All of the solid components are dissolved in the water, and once dissolved, the coating is applied to the tablet.

The coating forms a portion of the coated tablet. Typically, the coating is present in an amount ranging from 2 wt. % to 8 wt. %, for example 4 wt. % to 5 wt. %, based on the total weight of the uncoated tablet.

Unlike tablets having enteric coatings, where absorption of the SAMe or other hydroscopic material contained in the tablet is delayed until the coating is exposed to a pH above 5 to 6, the coating of the present disclosure can disintegrate over a range of pH's, both acidic and neutral. The ability of the coating to disintegrate in acidic pH's will allow for absorption of the SAMe or other hydroscopic material contained in the tablet quicker than in cases where an enteric coating is used. Further, the ability to rapidly disintegrate will result in less variability in absorption of the SAMe or other hydroscopic material into the blood stream.

A method of manufacturing the coating is also provided. The method includes combining the components used to form the coating described above, including at least the polymer film former, the moisture barrier, the calcium carbonate, and the plasticizer. During the process of manufacturing the coating, the combination of ingredients may develop a partially-hydrophobic layer of protection.

A method of manufacturing the coated tablet is also provided. The method includes applying the coating described above to an uncoated tablet containing the SAMe, milk thistle, and/or any other hydroscopic raw material. Various methods can be used to apply the coating to the uncoated tablet, for example, a traditional standard coating pan, a perforated pan system, or a fluidized system.

The coating provides several advantages, including delaying or preventing moisture from entering the coated tablet. The process of applying the coating of the present disclosure to a tablet is less complex than processes used to apply enteric coatings to tablets. The more complex process is needed for each agent in the enteric coating to achieve enteric properties. The enteric coating process exposes the SAMe, or other hydroscopic material contained in the tablet, to more moisture and higher temperatures. SAMe is susceptible to temperature and humidity, which causes degradation. The coating of the present disclosure can be applied to the tablet in less time, compared to the enteric coatings, which means less exposure of the sensitive materials to heat and moisture during the process.

### Experiment

A stress test was conducted on the coated tablets of the present disclosure to evaluate their performance and determine whether the coated tablets absorb an unacceptable amount of moisture. As stated above, SAMe is susceptible to temperature and humidity, which causes degradation. The stress test included exposing the coated tablets to a temperature of 50° C. for 5 days. The tablets were evaluated at 0 days, 3 days, and 5 days to compare degradation of the SAMe raw material.

The tablets were formulated to contain a minimum of 425 mg of the SAMe ion before stress testing. The specific SAMe material present in the coated tablets tested was S-Adenosyl Methionine (SAMe) Disulfate Tosylate which includes a minimum of 49% SAMe ion. The inventive and comparative tablets tested had the same composition, other than the coating.

The coating of the tablets according to the inventive example (Example 1) had the composition described above, specifically a combination of hydroxypropyl methyl cellulose, purified stearic acid, calcium carbonate, sunflower lecithin, triethyl citrate, and caramel color. The hydroxypropyl methyl cellulose was present in an amount of 0.7 to 2.8 weight percent (wt. %); the purified stearic acid was present in an amount of 0.6 to 2.4 wt. %; the calcium carbonate was present in an amount of 0.47 to 1.92 wt. %; the triethyl citrate and sunflower lecithin was present in a total amount of 0.2 to 0.8 wt. %; and the caramel color was present in an amount of 0.02 to 0.09 wt. %, based on the total weight of the uncoated tablet. All of the solid components of the coating system are dispersed in the water at certain percent solids, and once uniformly dispersed, the coating is applied to the tablet at the appropriate weight gain.

Two comparative coated tablets were also tested. The coating of the comparative coated tablets included methacrylic acid copolymer in an amount of 14.2 to 56.81 mg, titanium dioxide in an amount of 2.82 to 11.27 mg, anhydrous colloidal silica in an amount of 0.33 to 1.32 mg, talc in an amount of 3.12 to 12.48 mg, triethyl citrate in an amount of 3.29 to 13.16 mg. The inventive example composition and first comparative example composition were placed in high density polyethylene (HDPE) bottles. The test results are provided in Table 1 below and include the quantity of the SAMe ion (mg) present in the coated tablet after exposing the coated tablets to a temperature of 500 C for 5 days.

TABLE 1

| | | Comparative | Comparative |
|---|---|---|---|
| | Example 1 | Ex. 1 | Ex. 2 (2 lots) |
| Quantity of | 420 | 406 | 412 / 430 |
| SAMe ion (mg) | | | |

As shown in Table 1, the coated tablet of the present disclosure (Example 1) included 420 mg of the SAMe ion at the conclusion of the test. The coated tablet of comparative Example 1 included 406 mg of the SAMe ion at the conclusion of the test; and the two coated tablets of comparative Example 2 included 412 mg and 430 mg of the SAMe ion at the conclusion of the test. The test results indicate the coating of Example 1 had a similar if not better performance than the comparative coatings. Long term stability of the coating of example 1 was ongoing at 25° C./60% RH, 30° C./65% RH and 40° C./75% RH during the typical stability evaluation.

The foregoing descriptions of various embodiments of the disclosure are provided for purposes of illustration and are not intended to be exhaustive or limiting. Modifications or variations are also possible in light of the above teachings. The embodiments described above were chosen to enable one of ordinary skill in the art to utilize the disclosed disclosures in various embodiments and with various modifications as are suited to the particular use contemplated. All such modifications and variations are within the scope of the disclosure.

What is claimed is:

1. A coated tablet for a human or animal, comprising:
a tablet including SAMe (S-Adenosylmethionine), milk thistle, and/or any other hydroscopic raw material; and
a coating including a polymer or resin, stearic acid, and calcium carbonate.

2. The coated tablet of claim 1, wherein the stearic acid is present in an amount of 0.6 to 2.4 wt. %, and the calcium carbonate is present in an amount of 0.47 to 1.92 wt. %, based on the total weight of the uncoated tablet.

3. The coated tablet of claim 1, wherein the coating is partially hydrophobic.

4. A coated tablet for a human or animal, comprising:
a tablet including SAMe (S-Adenosylmethionine), milk thistle, and/or any other hydroscopic raw material; and
a coating including a polymer or resin, a moisture barrier, calcium carbonate, and a plasticizer.

5. The coated tablet of claim 4, wherein the polymer or resin is present in an amount of 0.7 to 2.8 weight percent (wt. %); the moisture barrier is present in an amount of 0.6 to 2.4 wt. %; the calcium carbonate is present in an amount of 0.47 to 1.92 wt. %; and the plasticizer is present in a total amount of 0.2 to 0.8 wt. %, based on the total weight of the uncoated tablet.

6. The coated tablet of claim 4, wherein the moisture barrier is purified stearic acid.

7. The coated tablet of claim 4, wherein the polymer or resin is hydroxypropyl methyl cellulose, the moisture barrier is purified stearic acid, and the plasticizer includes triethyl citrate.

8. The coated tablet of claim 7, wherein the plasticizer further includes lecithin.

9. The coated tablet of claim 4 further including a color agent.

10. The coated tablet of claim 9, wherein the polymer or resin is hydroxypropyl methyl cellulose, the moisture barrier is purified stearic acid, the plasticizer includes triethyl citrate and lecithin, and the color agent includes caramel.

11. A method of manufacturing a coated tablet for a human or animal, the method comprising the steps of:
applying a coating to a tablet;
wherein the tablet includes SAMe (S-Adenosylmethionine), milk thistle, and/or any other hydroscopic raw material; and
the coating includes a polymer or resin, stearic acid, and calcium carbonate.

12. The method of claim 11, wherein the stearic acid is present in an amount of 0.6 to 2.4 wt. %, and the calcium carbonate is present in an amount of 0.47 to 1.92 wt. %, based on the total weight of the uncoated tablet.

13. The method of claim 11, wherein the coating is partially hydrophobic.

14. A method of manufacturing a coated tablet for a human or animal, the method comprising the steps of:
applying a coating to a tablet;
wherein the tablet includes SAMe (S-Adenosylmethionine), milk thistle, and/or any other hydroscopic raw material; and
the coating includes a polymer or resin, a moisture barrier, calcium carbonate, and a plasticizer.

15. The method of claim 14, wherein the polymer or resin is present in an amount of 0.7 to 2.8 weight percent (wt. %); the moisture barrier is present in an amount of 0.6 to 2.4 wt. %; the calcium carbonate is present in an amount of 0.47 to 1.92 wt. %; and the plasticizer is present in a total amount of 0.2 to 0.8 wt. %, based on the total weight of the uncoated tablet.

16. The method of claim 14, wherein the moisture barrier is purified stearic acid.

17. The method of claim 14, wherein the polymer or resin is hydroxypropyl methyl cellulose, the moisture barrier is purified stearic acid, and the plasticizer includes triethyl citrate.

18. The method of claim 17, wherein the plasticizer further includes lecithin.

19. The method of claim 14 further including a color agent.

20. The method of claim 19, wherein the polymer or resin is hydroxypropyl methyl cellulose, the moisture barrier is purified stearic acid, the plasticizer includes triethyl citrate and lecithin, and the color agent includes caramel.
